(19) United States
(12) Patent Application Publication
(10) Pub. No.: US 2002/0045572 A1
(43) Pub. Date: Apr. 18, 2002

(54) Method of treating the syndrome of type 2 diabetes in humans

(75) Inventor: Anton H. Clemens, Madison, WI (US)

Correspondence Address: MICHAEL BEST & FRIEDRICH, LLP, ONE SOUTH PINCKNEY STREET, P O BOX 1806, MADISON, WI, 53701

(73) Assignee: CPD, LLC, Madison, WI (US)

(21) Appl. No.: 09/878,751

(22) Filed: Jun. 11, 2001

Related U.S. Application Data
(63) Continuation-in-part of application No. 09/638,930, filed on Aug. 15, 2000.

(51) Int. Cl. A61K 38/28; A61K 31/485

(52) U.S. Cl. 514/3; 514/282

(57) ABSTRACT

The invention provides a method of treating a human suffering from the Syndrome of Type 2 Diabetes by administering, by a pharmaceutically effective mode, a drug composition having an opioidergic agent including opiates having μ-agonist activity, opiates having κ antagonist activity or a combination thereof and an insulin secretagogue.

### CROSS-REFERENCE TO RELATED APPLICATION

This application is a continuation-in-part of and claims priority to U.S. application Ser. No. 09/638,930 filed on Aug. 15, 2000.

### BACKGROUND OF THE INVENTION

Type 2 Diabetes is a major cause of death in the industrialized world. A wide variety of chemical and physical abnormalities are associated with Type 2 Diabetes, which are a consequence of, and associated with, imbalances in fuel metabolism and impaired hepatic fuel processing. The typical American and Western European diet, i.e. fuel intake, consists of 40-45% carbohydrates, 40% fat and 15-20% protein. Type 2 Diabetes includes elevations in fasting blood glucose, gluconeogenesis and glucose production, in spite of significant increases in fasting insulin and C-peptide concentrations. Hepatic gluconeogenesis is the formation of glucose, particularly by the liver, from non-carbohydrate sources like pyruvate, lactate, odd-chain fatty and amino acids, and glucose production (GP) is the formation of glucose from carbohydrate sources, e.g. glycogen. The underlying insulin resistance (IR) associated with Type 2 Diabetes also contributes to increases in lipogenesis. Typically associated with lipogenesis is dislipidemia, which is characterized by increases in levels of fasting free fatty acid (FFA), fasting triglycerides (TG) and total cholesterol concentrations, increases in levels of fasting LDL-cholesterol, decreases in levels of fasting HDL-cholesterol, and an increased LDL/HDL ratio. In addition, lipogenesis leads to increases in body weight and increases in systolic and diastolic blood pressure.

Type 2 Diabetes represents a syndrome of various, in part sequential, disease states. Its pathophysiology slowly progresses through a long period of successive abnormalities: a) Insulin Resistance (IR), which in association with beta-cell dysfuntion mediated excessive hepatic gluconeogenesis (GNG) and Glucose Production (GP), leads to b) Impaired Fasting Glucose (IFG) and, in turn, to c) Impaired Glucose Tolerance (IGT) and eventually to the clinical form of d) Non-Insulin Dependent Diabetes Mellitus (NIDDM). IR is characterized as a state in which a normal amount of insulin produces a subnormal biological response in carbohydrate metabolism. IR tends to remain active throughout the entire pathophysiology of this syndrome. Beta-cell dysfunction is characterized by a gradual disappearance of the so-called first phase insulin secretion. In order to normalize blood glucose levels, affected subjects require above-normal levels of second, or proportional phase insulin release to cope with excessive post-prandial glucose excursions and to compensate for their insulin resistance.

Compared to Type 1 Diabetes (juvenile diabetes), the Type 2 Diabetes syndrome, particularly its NIDDM form, is characterized by relatively inadequate endogenous insulin concentrations. However, insulin concentrations in Type 2 diabetics may, in fact, be higher than in the normal population.

Non-diabetics experience a biphasic insulin response, i.e. an acute phase and a proportional phase. The acute phase, also referred to as first phase insulin release, is prompted by a rise in blood glucose; the proportional, second phase is characterized by a more sustained insulin release type 2 diabetics experience an absence of the acute phase insulin release governed by the level of blood glucose and follows the acute phase. Contrastly, Type 2 diabetics suffer from impaired first phase β-cell insulin secretion, or the absence thereof.

Persons afflicted with Type 2 Diabetes also experience a rise in GNG and GP during the early morning before waking. Compounded with impaired insulin secretion, the rise in GNG and GP results in elevated fasting glucose levels. Many different treatments have been sought to try to address the impaired insulin secretion, i.e. Beta-Cell Dysfunction (BCD), and increased glucose levels; a pharmacologic restoration of the acute, first phase insulin release, the primary defect of BCD has, to date, proven to be an elusive undertaking. BCD may play an early and very key role in the development of Type 2 Diabetes, and even of obesity and Insulin Resistance (IR) (Balasubramanyan, Ashok, Report on 60^{th} Scientific Sessions of the ADA, Jun. 10, 2000).

Restoration of the first phase insulin release in clinical research by the Artificial Beta Cell (Clemens, A. H.; U.S. Pat. No. 4,055,175) in Type 1 diabetics has been used to return a Type 1 diabetic patient's hepatic capacity to oxidize and store carbohydrates to normal. (Foss, M. C., Vlachokoska, V., Cunningham, L. N. and Aoki, T. T.; Diabetes, 31:46-52). Type 2 diabetics as well as IGT, overweight and obese subjects are characterized by an impaired β-cell function (islet dysfunction), with relatively intact second, proportional phase insulin release capacity, but defective or lacking acute, first phase insulin release (Pfeifer, M. A., Halter, J. B., Porte D., Jr.; Am J Med, 70:579-88). They are also characterized by impaired hepatic carbohydrate oxidation and storage (Felber, J.-P., Meyer, H. U., Curchod, B., Maeder, E., Pahud, P., and Jequier, E.; Metabolism, 30,2; 184-189; and Diabetologia, 20: 39-44).

Currently available methods for treatment of Type 2 Diabetes can be classified as Insulin Secretagogues, Insulin Sensitizers, agents to reduce Gluconeogenesis (GNG) and Glucose Production (GP), and agents to modify the absorption of carbohydrates from the intestine.

Sulfonylureas, belonging to the Insulin Secretagogues, represent the traditional, and most common category of oral antidiabetic drugs; they stimulate Insulin Secretion with or without the presence of elevated blood glucose levels and are, therefore, known for their risk of producing hypoglycemia and for their failure to restore physiologic beta-cell functions and first phase insulin release.

Metformin hydrochloride, a non-sulfonylurea type antihyperglycemic agent of the Biguanide family, improves glucose tolerance in Type 2 diabetic subjects, primarily by decreasing hepatic gluconeogenesis and glucose production (Edelman, S. V.: Clinical Diabetes, 1998: 16,1: 37-40). However, metformin does not restore first phase insulin secretion. Major side effects of using metformin include potential lactic acidosis and negative impact on liver and kidney function resulting from the requisite massive therapeutic doses. Therefore, metformin is contraindicated for patients with hepatic or renal insufficiency, which is aggravated by the fact that a typical daily dose ranges between 1,500 and 2,500 mg.

As a result, it is desirable to provide a new method for treating the GNG and GP symptoms of Type 2 Diabetes. In addition, a method is desired wherein Type 2 Diabetes in a human can be treated by restoring first phase insulin release. A new method for decreasing the elevated fasting glucose levels in patients suffering from Type 2 Diabetes is also desired. In other words, a new treatment is desirable that lowers the high glucose levels resulting from rises in GNG and GP, and impaired insulin secretion in patients afflicted with Type 2 Diabetes.

### SUMMARY OF THE INVENTION

The invention provides an improved method of treating a human suffering from the Syndrome of Type 2 Diabetes. The method includes administering, by a pharmaceutically effective mode, a drug composition comprising an opioidergic agent and an insulin secretagogue. More particularly, the opioidergic agent may be an opiate having μ agonist activity.

The invention further provides a method of treating a human suffering from the Syndrome of Type 2 Diabetes by administering, by a pharmaceutically effective mode, a drug composition comprising an opiate having μ-agonist activity, an opiate having κ-antagonist activity and an insulin secretagogue.

The invention further provides a method of treating a human suffering from the Syndrome of Type 2 Diabetes by administering, by a pharmaceutically effective mode, a drug composition comprising an opiate having κ antagonist activity and an insulin secretagogue.

The invention also provides a method for treating the Syndrome of Type 2 Diabetes, which includes various progressive disease states from IR, GNG, GP, IFG and IGT to the clinical form of Type 2 Diabetes, as well as excessive lipogenesis and dyslipidemia.

The invention also provides an improved first pass insulinization of the liver, resulting in a restoration of enzyme functions involved in hepatic carbohydrate oxidation and storage.

Before embodiments of the invention are explained in detail, it is to be understood that the invention is not limited in its application to the details of the composition and concentration of components set forth in the following description. The invention is capable of other embodiments and of being practiced or being carried out in various ways. Also, it is understood that the phraseology and terminology used herein is for the purpose of description and should not be regarded as limiting. Other features and advantages of the invention will become apparent to those skilled in the art upon review of the following detailed description and claims.

The patents, references and articles cited herein are fully incorporated by reference.

### BRIEF DESCRIPTION OF DRAWINGS

FIG. 1 is a graph showing the daily blood glucose profile of a 72-year subject afflicted with Type 2 Diabetes after treatment with an opioidergic drug composition combined with an insulin secretagogue. The blood glucose (BG) of the subject was measured in mg/dL over several time intervals measured in hours (h).

### DETAILED DESCRIPTION

As used herein, the term “insulin secretagogue” is meant to refer to any drug composition which stimulates, participates in the stimulation of, or potentiates, the secretion of insulin by the pancreatic beta-cells. Insulin secretagogues include insulinotropic agents and insulin secretion or release potentiators. Examples are provided below.

As used herein, the terms “opioids,” “opioid agonists,” “opiate agonists,” “opiates having agonist activity” and “agonists” are meant to refer to opioid-like substances, natural or synthetic, that bind to centrally and/or peripherally located opioid receptors to produce an agonist action.

As used herein, the terms “μ”, “δ” and “κ” refer to the specific types of opiate receptors of cells such as brain, spinal cord and large intestine.

As used herein, the terms “opiates having μ agonist activity,” “opioids having μ agonist activity” and “μ agonists” are meant to refer to opioid-like substances, natural or synthetic, that bind to the μ opiate receptor to produce an agonist action.

As used herein, the terms “opioid antagonists,” “opiate antagonists,” “anti-opioids,” “opiates having antagonist activity” and “antagonists” are meant to refer to opioid-like substances that bind to opioid receptors, but produce little or no agonist activity.

As used herein, the terms “opiates having κ antagonist activity,” “opioids having κ antagonist activity” and “κ antagonists” are meant to refer to opioid-like substances that bind to the κ opiate receptor, but produce little or no agonist activity.

As used herein, the terms “opioidergic agent” and “opioidergic drug composition” are meant to refer to any of the above opioid-like substances as well as combinations thereof.

“Pharmaceutically effective modes” are meant to include, but not be limited to the application of a drug composition as a solution in an innocuous pharmaceutically acceptable solvent, as an emulsion, as a suspension, as a dispersion in suitable carriers, as a patch or in the form of pills or capsules with solid carriers, and other such methods well-known in the art. The formulations of this invention may include pharmaceutically acceptable excipients such as stabilizers, anti-oxidants, binders, coloring agents, emulsifiers, and other such excipients well-known in the art. The drugs and drug compositions comprising the agonists and antagonists described above and below, may be administered in any pharmaceutically effective mode.

During the investigations into, and development of, non-addictive morphine based analgesics, typically requiring a combination of agonistic and antagonistic actions at various opiate receptor sites, i.e.μ, δ and κ receptors, a variety of so-called antagonists have evolved as by-products, and some of these narcotic antagonists, or anti-opioids, have been shown to have potential in the treatment of a variety of disease conditions.

U.S. Pat. No. 4,619,936 discloses pharmaceutical compositions containing (5α, 6α)7,8-didehydro-4,5-epoxy-17-(2-propanyl)-morphinano-3,6-diol for the purpose of appetite reduction.

U.S. Pat. No. 5,727,570 discloses a method of treatment of humans suffering hyperlipidimia from by administering a drug composition selected from a group consisting of opiate antagonists and drugs which substantially equally reduce the amount of catecholamines bound to catecholamine binding sites.

U.S. Pat. No. 5,878,750 discloses a method of treating humans suffering from the Coronary Heart Disease Syndrome by administering a drug composition selected from the group of opiate antagonists or anti-opioids and drugs which substantially equally reduce the amounts of catecholamines bound to all catecholamine binding sites.

Drugs which are useful in the methods of the present invention, i.e. methods for treating human suffering from the Syndrome of Type 2 Diabetes, include centrally or peripherally acting opioid compositions, e.g. opiates having μ agonist activity. In addition, effective drug compositions include μ agonists in combination with selective κ antagonists. Drug compositions including pure non-selective antagonists with pronounced κ antagonist characteristics can also be utilized in these methods. Drug compositions comprising mixed μ-agonist/κ-antagonists can also be employed in the methods. Examples of different agonists and antagonists are listed below, and are not meant to limit the scope of drug compositions which can be administered to treat Type 2 Diabetes.

Bi-directional effects of opioids are known to exist, in particular between μ and κ agonists, such as μ agonists producing euphoria and κ agonists producing the opposite, namely disphoria. Conversely, μ antagonists can antagonize euphoria and enhance the effect of the κ agonist, while the κ antagonist can produce or enhance euphoria. Examples of this phenomenon also include the opposing effects of μ and κ opiates in motivational processes (Herz A.: NIDA Res Monogr 90:17-26), or in opioid reward mechanisms (Herz A.: Can J Physiol Pharmacol 76,3:252-8), and other μ-opposing actions of the κ-opioid receptor (Pan Z. Z.: Trends Pharmacol Sci; 19,3: 94-8).

Opioids having selective or predominant κ-antagonist activity include, but are not limited to, nor-binaltorphine, (Portoghese, P. S., Lipkowski, A. W., Takemori, A. E.; Life Sciences 40: 1287-92); guanidylated naltrindole (GNTI), (Jones R. M., Hjorth, A. S., Schwartz, T. W., and Portoghese, P. S.; Journal of Medicinal Chemistry 41,25: 4911-4), (−)-(1R,5R,9R)-5,9-diethyl-2-(3-furylmethyl)-2-hydroxy-6,7-benzomorphan (MR 2266) (Merz, H., Langbein, A., Stockhaus, K., Walther, G., & Wick, H.; Advances in biochemical psychopharmacology, Vol 8: 91-107), a triethylenedioxy derivative of B-naltrexamine (TENA), (Portoghese, P. S., Takemori, A. E.; Life Sciences 36: 801-5) and buprenorphine.

Opioids having selective or predominant μ agonist activity include, but are not limited to, dihydromorphine, morphine, hydromorphone, methadone, fentanyl, sufentanyl, buprenorphine, demorphine, codeine, ethylmorphine, etonitazene, hydrocodone, levorphanol, norcodeine, normorphine and oxycodone. Most opioids pass the Blood Brain Barrier (BBB) and are, therefore both, centrally and peripherally active, i.e. they can act upon CNS sites as well as peripheral sites, such as the gut and hormone producing glands, including the endocrine pancreas and the adrenal medulla. Some opioids, e.g. loperamide, do not pass the BBB and are, therefore, only peripherally active with little or no CNS effect. Since drug addiction generally requires a central effect, peripherally acting opioid agonists are typically not addictive and generally not ‘scheduled’ as narcotics.

Buprenorphine is a mixed agonist-antagonist with high affinity at the μ opiate receptor with partial agonist activity, and at the κ receptor with antagonist activity. Because of its κ receptor antagonist activity and low partial μ activity it will produce minimal and perhaps clinically insignificant physical dependence, and has been used as an effective analgesic for the treatment of moderate to severe pain and of opioid dependence. (Lewis J. W.: Drug and Alcohol Dependence; 14:363-372). Elevations in cortisol and glucose, caused by surgical stress, have been observed to decline following the administration of buprenorphine to treat analgesia during and following total hip replacement (McQuay H. J. et. al.: Br J Anaesth; 52:1013-19).

Loperamide is a synthetic opioid used for the treatment of diarrhea, which is more effective and safer than other opioid drugs in the treatment of diarrhea of various causes (Ruppin H: Acta Physiol Scand; 127,3:275-9) Loperamide is a ‘non-scheduled’ opioid with μ agonist activity as opposed to most other opioid agonists which are listed as ‘controlled substances’. Loperamide is reported to raise blood glucose concentrations at dose levels required for the acute treatment of diarrhea (Caldara R. et. al.: Eur J Clin Pharmacol; 21,3:185-8), and has been used in the “Loperamide test”: a simple and highly specific screening test for hypercortisolism in children and adolescents” (Buzi F. et. al.: Acta Paediatr; 86,11: 1177-80).

Insulin secretagogues include, but are not limited to, sulphonylureas, insulinotropic agents and drugs known to potentiate insulin release. Insulin secretagogues include, but are not limited to tolbutamide, chlorpropamide, glimepiride, glipizide, and glyburide, (all listed in Merck Index, 12^{th} Edition, 1996), repaglinide, (Raskin P et.al.: Diabetes Care 23[7]:979-83), meglitinides, morphilinoguanide and pramlintide (Evans A J and Kreutz A J: Drugs RD 2[2]:75-94), acetylcholine (Tae Niwa et. al.: Diabetes 47: 1699-1706), muscarinic agonists, e.g.: carbachol (Hiriart M and Ramirez-Medeles M C: Mol Cell Endocrinol 93[1]: 63-9) and bethanechol (Physicians Desk Reference [PDR]), beta-L-glucose pentaacetate, (Malaisse W J et.al.: American Journal of Physiology 276[6/1]: E993-E1006), chiro-inositol (Larner J. et.al. U.S. Pat. No. 5,428,066), myo-inositol (Szkudelski T and Kandulska K.: Arch. Physiol. Biochem. 107[4]:334-7), and Incretin Factors, e.g.: GIP (Kindmark H et.al.:J Clin Endocrinol Metab 86[5]:2015-9), GLP-1 and Extendin-4 (Parkes D G et.al.: Metabolism 50[5]:583-9).

The invention provides the supplementation of the opioidergic method of treating the Syndrome of Type 2 Diabetes with the concurrent administration of insulin secretagogues, including, but not limited to sulphonylureas, insulinotropic agents and insulin release potentiators, which include but are not limited to, acetylcholine agonists, beta-L-glucose pentacetate, glucagon-like peptide-1 (GLP-1), Glucose-Dependent-Insulinotropic Hormone (GIP), extendin-4 and chiro-inositol and related structures. Similar methods can be used to treat various progressive disease states included within the Syndrome of Type 2 Diabetes, which include, but are not limited to, IR, GNG, GP, IFG and IGT as well as excessive lipogenesis and dyslipidemia.

Non-glucose dependent insulin secretagogues, such as sulphonylureas, stimulate insulin secretion regardless of the blood glucose level and are, therefore, prone to produce potentially deleterious hypoglycemia, particularly so, because such insulin secretagogues primarily increase the second phase insulin release without any improvement in first phase insulin secretion. This invention of administering, by a pharmaceutically effective mode, opioidergic drug compositions comprising opiates having μ agonist and/or κ antagonist activity, in combination with non-glucose dependent insulin secretagogues, such as sulphonylureas, can confer a more glucose dependent, bi-phasic insulin release pattern on such secretagogues, and significantly reduce the likelihood of producing hypoglycemia.

### EXAMPLE

The example is being described for purely illustrative purposes, and is in no way meant to limit the scope of the invention.

The daily blood glucose profile of a 72 year old subject with Type 2 Diabetes and dislipidemia was monitored following the early morning oral administration of 0.15 mg Hydrocodone, a centrally acting μ-agonist in combination with 1.0 mg Glipizide, a sulfonylurea type insulin secretagogue. The glucose profile of the subject was monitored on the second day of treatment. Sufficient time was allowed between the earlier treatment method to eliminate any carry-over effect.

Treatment 1
(-O-) 110 drug treatment.
Breakfast: 08:40-09:00; lunch: 12:25-12:50; dinner: 18:40-19:00

| | | | | | | |
|---|---|---|---|---|---|---|
| time [h] | 08:10 | 10:00 | 12:20 | 14:00 | 18:30 | 20:00 |
| BG [mg/dL] | 132 | 156 | 104 | 150 | 98 | 117 |

Treatment 2
(-▪-) 0.15 Hydrocodone, a centrally acting μ-agonist in combination with 1.0 mg
Glipizide, administered 2 hours before breakfast.
Breakfast 09:00-09:15, Lunch 12:10-12:30, Dinner 19:20-19:50

| | | | | | | | | | | | | | | |
|---|---|---|---|---|---|---|---|---|---|---|---|---|---|---|
| time [h] | 08:45 | 09:00 | 09:15 | 09:50 | 10:35 | 11:05 | 12:10 | 12:30 | 13:00 | 14:05 | 19:25 | 19:50 | 20:25 | 20:55 |
| BG | 103 | 102 | 102 | 108 | 116 | 104 | 109 | 100 | 108 | 94 | 92 | 88 | 105 | 89 |
| [mg/dL] | | | | | | | | | | | | | | |

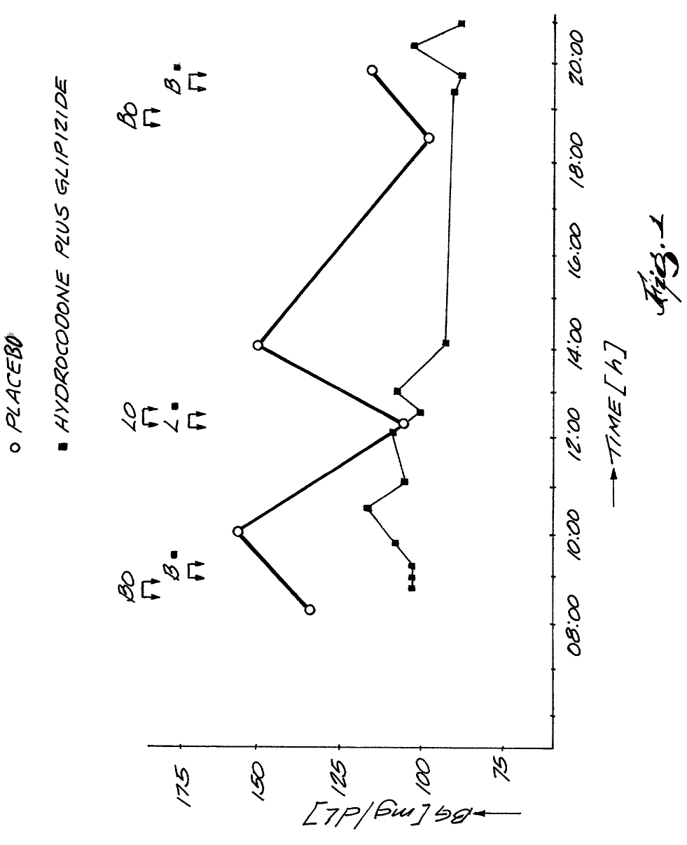

FIG. 1 and the supporting data demonstrate that post prandial peaks in glucose concentration have been virtually normalized with the administration of 0.15 mg of hydrocodone in combination with 1.0 mg of the insulin secretagogue glipizide (-▪-). The administration of the μ-agonist in combination with the insulin secretagogue also results in very significant increases in first phase insulin secretion, as can be derived from the pronounced decreases of blood glucose concentrations throughout the intake of meals, and from the reduced duration and excursion of the post-prandial glucose peaks, compared to placebo.

The application of any of the μ-agonists, according to this invention, can be enhanced, or supplemented by selective κ-antagonists, non-selective antagonists having κ0 antagonist activity, or by mixed μ-agonist/κ-antagonists. It is also a subject of this invention to use a mixed μ-agonist/κ-antagonists as a single molecular entity.

The opioidergic agents useable in the present invention may be single substances or may also be a combination of opioids, as long as their respective pharmacodynamics are, or have been rendered, compatible, in order to avoid significant variations in their respective, and combined, biologic effectiveness throughout their decline in biological action.

The effective dose of loperamide is, typically, less than 0.5 mg per day, or smaller by more than an order of magnitude than the typical dose required for the treatment of diarrhea, and the effective dose for hydrocodone is, typically, less than 1 mg. The effective dose for these and other μ-agonists, or single- or multi-molecular mixed μ-agonist/κ-antagonist as well as compositions insulin secretagogues, may vary depending upon factors such as the (patho-) physiology of the subject being treated, receptor binding, the absorption rate, bio-availability, excretion rate and the rate of metabolism of the drug.

The pharmacokinetic compatibility between the opioidergic agent and the insulin secretagogue is less critical, but the preferred biologic half life of the insulin secretagogue is less than 4 hours. The preferred method of administration is such that the peak plasma concentration of the drug combination occurs before or during breakfast.

The effective dose for the supplementary insulin secretagogues is, typically, equal or less than the dose for such agents used in monotherapy.

I claim:

1. A method of treating a human suffering from the Syndrome of Type 2 Diabetes comprising administering, by a pharmaceutically effective mode, of a drug composition comprising:
an opioidergic agent; and
an insulin secretagogue.

2. The method of claim 1, wherein the opioidergic agent is an opiate having μ agonist activity.

3. The method of claim 2, wherein the opioidergic agent includes at least one of the following:
i) dihydromorphine;
ii) morphine;
iii) hydromorphone;
iv) methadone;
v) fentanyl;
vi) sufentanyl;
vii) buprenorphine;
viii) demorphine;
ix) codeine;
x) ethylmorphine;
xi) etonitazene;
xii) hydrocodone;
xiii) levorphanol;
xiv) norcodeine;
xv) normorphine; and
xvi) oxycodone.

4. The method of claim 2, wherein the opiate having μ-agonist activity is a centrally acting μ-agonist.

5. The method of claim 2, wherein the opiate having μ-agonist against activity is a peripherally acting μ-agonist.

6. The method of claim 5, wherein the peripherally acting against is loperamide.

7. The method of claim 2, wherein the Syndrome of Type 2 Diabetes includes Impaired Fasting Glucose (IFG).

8. The method of claim 2, wherein the Syndrome of Type 2 Diabetes includes Impaired Glucose Tolerance (IGT).

9. The method of claim 2, wherein the Syndrome of Type 2 Diabetes includes impaired hepatic fuel processing.

10. The method of claim 9, wherein the impaired hepatic fuel processing includes control of carbohydrate oxidation and storage.

11. The method of claim 2, wherein in the Syndrome of Type 2 Diabetes includes excessive endogenous gluconeogenesis (GNG).

12. The method of claim 2, wherein the Syndrome of Type 2 Diabetes includes excessive endogenous glucose production (GP).

13. The method of claim 2, wherein the Syndrome of Type 2 Diabetes includes lipogenesis excess and dislipidemia.

14. The method of claim 2, wherein the Syndrome of Type 2 Diabetes includes impaired first phase β-cell insulin secretion.

15. The method of claim 2, wherein the Syndrome of Type 2 Diabetes includes insulin resistance (IR).

16. The method of claim 1, wherein the insulin secretagogue includes at least one of the following:
i. sulphonylureas;
ii. tolbutamide;
iii. chlorpropamide;
iv. glimepiride;
v. glipizide;
vi. glyburide;
vii. meglitinides;
viii. repaglinide;
ix. pramlintide;
x. morphilinoguanide;
xi. acetylcholine;
xii. muscarinic agonists;
xiii. carbachol;
xiv. bethanechol;
xv. beta-L-glucose pentaacetate;
xvi. chiro-inositol;
xvii. myo-inositol;
xviii. GIP;
xix. GLP-1; and
xx. Extendin-4.

17. The method of claim 1, wherein the insulin secretagogue is a non-glucose dependent insulin secretagogue, the method producing insulin release patterns capable of attaining glucose dependent, bi-phasic release characteristics with reduced likelihood of producing hypoglycemia.

18. The method of claim 17, wherein the insulin secretagogue is sulphonylurea.

19. A method of treating a human suffering from the Syndrome of Type 2 Diabetes comprising administering, by a pharmaceutically effective mode, a drug composition comprising:
an opiate having μ-agonist activity;
an opiate having κ-antagonist activity; and
an insulin secretagogue.

20. The method of claim 19, wherein the drug composition comprises a single molecular entity.

21. The method of claim 20, wherein the drug composition comprises buprenorphine.

22. The method of claim 19, wherein the drug composition comprises a combination of molecular entities.

23. The method of claim 19, wherein the drug composition includes at least one of the following:
i) dihydromorphine;
ii) morphine;
iii) hydromorphone;
iv) methadone;
v) fentanyl;
vi) sufentanyl;
vii) buprenorphine;
viii) demorphine;
ix) codeine;
x) ethylmorphine;
xi) etonitazene;
xii) hydrocodone;
xiii) levorphanol;
xiv) norcodeine;
xv) normorphine; and
xvi) oxycodone.

24. The method of claim 19, wherein the drug composition comprises a centrally acting μ-agonist.

25. The method of claim 19, wherein the drug composition comprises a peripherally acting μ-agonist.

26. The method of claim 25, wherein the peripherally acting μ-agonist is loperamide.

27. The method of claim 19, wherein the drug composition includes at least one of the following:
i) nor-binaltorphine;
ii) (−)-(1R,5R,9R)-5,9-diethyl-2-(3-furylmethyl)-2-hydroxy-6,7-benzomorphan (MR 2266);
iii) a triethylenedioxy derivative of B-naltrexamine (TENA); and
iv) guanidylated naltrindole (GNTI).

28. The method of claim 19, wherein the Syndrome of Type 2 Diabetes includes Impaired Fasting Glucose (IFG).

29. The method of claim 19, wherein the Syndrome of Type 2 Diabetes includes Impaired Glucose Tolerance (IGT).

30. The method of claim 19, wherein the Syndrome of Type 2 Diabetes includes impaired fuel processing.

31. The method of claim 30, wherein the impaired hepatic fuel processing includes control of carbohydrate oxidation and storage.

32. The method of claim 19, wherein the Syndrome of Type 2 Diabetes includes insulin resistance (IR).

33. The method of claim 19, wherein in the Syndrome of Type 2 Diabetes includes excessive gluconeogenesis (GNG).

34. The method of claim 19, wherein the Syndrome of Type 2 Diabetes includes excessive endogenous glucose production (GP).

35. The method of claim 19, wherein the Syndrome of Type 2 Diabetes includes lipogenesis excess and dislipidemia.

36. The method of claim 19, wherein the Syndrome of Type 2 Diabetes includes impaired first phase β-cell insulin secretion.

37. The method of 19, wherein the insulin secretagogue includes at least one of the following:
i. sulphonylureas;
ii. tolbutamide;
iii. chlorpropamide;
iv. glimepiride;
v. glipizide;
vi. glyburide;
vii. meglitinides;
viii. repaglinide;
ix. pramlintide;
xi. morphilinoguanide;
xii. acetylcholine;
xiii. muscarinic agonists;
xiv. carbachol;
xv. bethanechol;
xvi. beta-L-glucose pentaacetate;
xvii. chiro-inositol;
xviii. myo-inositol;
xix. GIP;
xx. GLP-1; and
xxi. Extendin-4;

38. The method of claim 19, wherein, the insulin secretagogue is a non-glucose dependent insulin secretagogue, the method producing insulin release patterns capable of attaining glucose dependent, bi-phasic release characteristics with reduced likelihood of producing hypoglycemia.

39. The method of claim 38, wherein the insulin secretagogue is sulphonylurea.

40. A method of treating a human suffering from the Syndrome of Type 2 Diabetes comprising administering, by a pharmaceutically effective mode, a drug composition comprising:
an opiate having κ antagonist activity; and
an insulin secretagogue.

41. The method of claim 40, wherein the opiate having κ antagonist activity includes at least one of the following:
i) nor-binaltorphine;
ii) (−)-(1R,5R,9R)-5,9-diethyl-2-(3-furylmethyl)-2-hydroxy-6,7-benzomorphan (MR 2266);
iii) a triethylenedioxy derivative of B-naltrexamine (TENA); and
iv) guanidylated naltrindole (GNTI).

42. The method of claim 40, wherein the Syndrome of Type 2 Diabetes includes Impaired Fasting Glucose (IFG).

43. The method of claim 40, wherein the Syndrome of Type 2 Diabetes includes Impaired Glucose Tolerance (IGT).

44. The method of claim 40, wherein the Syndrome of Type 2 Diabetes includes impaired fuel processing.

45. The method of claim 44, wherein the impaired fuel processing includes control of carbohydrate oxidation and storage.

46. The method of claim 40, wherein in the Syndrome of Type 2 Diabetes includes excessive gluconeogenesis (GNG).

47. The method of claim 40, wherein the Syndrome of Type 2 Diabetes includes excessive endogenous glucose production (GP).

48. The method of claim 40, wherein the Syndrome of Type 2 Diabetes includes lipogenesis excess and dyslipidemia.

49. The method of claim 40, wherein the Syndrome of Type 2 Diabetes includes impaired first phase β-cell insulin secretion.

50. The method of claim 40, wherein the Syndrome of Type 2 Diabetes includes insulin resistance (IR).

51. The method of 40, wherein the insulin secretagogue includes at least one of the following:
i. sulphonylureas;
ii. tolbutamide;
iii. chlorpropamide;
iv. glimepiride;
v. glipizide;
vi. glyburide;
vii. meglitinides;
viii. repaglinide;
ix. pramlintide;
xi. morphilinoguanide;
xii. acetylcholine;
xiii. muscarinic agonists;
xiv. carbachol;
xv. bethanechol;
xvi. beta-L-glucose pentaacetate;
xvii. chiro-inositol;
xviii. myo-inositol;
xix. GIP;
xx. GLP-1; and
xxi. Extendin-4.

52. The method of claim 40, wherein, the insulin secretagogue is a non-glucose dependent insulin secretagogue, the method producing insulin release patterns capable of attaining glucose dependent, bi-phasic release characteristics with reduced likelihood of producing hypoglycemia.

53. The method of claim 52, wherein the insulin secretagogue is sulphonylurea.
